(19) United States
(12) Patent Application Publication
(10) Pub. No.: US 2017/0027508 A1
(43) Pub. Date: Feb. 2, 2017

(54) BIOLOGICAL INFORMATION DETECTING DEVICE

(71) Applicant: SEIKO EPSON CORPORATION, Tokyo (JP)

(72) Inventors: Tetsu NAKAYAMA, Matsumoto-shi (JP); Hideto YAMASHITA, Suwa-shi (JP); Yuichi TAKEUCHI, Shiojiri-shi (JP); Yasutomo TAKAHASHI, Matsumoto-shi (JP)

(73) Assignee: SEIKO EPSON CORPORATION, Tokyo (JP)

(21) Appl. No.: 15/224,529

(22) Filed: Jul. 30, 2016

(30) Foreign Application Priority Data
Jul. 30, 2015 (JP) 2015-150678

(51) Int. Cl. A61B 5/00; A44C 5/00

(52) U.S. Cl. CPC A61B 5/681; A44C 5/0007

(57) ABSTRACT

A biological information detecting device includes: a biological information detector which detects biological information of a subject; and a band. In the band, a first surface which is on the side of the subject when the band is worn is wave-shaped in a cross section taken along a long-side direction of the band.

### CROSS-REFERENCES TO RELATED APPLICATIONS

This application claims priority to Japanese Patent Application No. 2015-150678, filed Jul. 30, 2015, the entirety of which is herein incorporated by reference.

### BACKGROUND

### 1. Technical Field

The present invention relates to a biological information detecting device or the like.

### 2. Related Art

Recently, there is an increasing need for sensing biological information not only with traditional activity monitors but also with sporting devices used in high-intensity movements such as a running watch. Bands used to fasten such an activity monitor, watch (timepiece) or the like on a subject's armor the like, for example, have been invented as well. Such traditional techniques include the techniques disclosed in JP-A-2010-110634 and JP-A-07-213312, for example.

In the case of the sporting device used in high-intensity movements such as a running watch, the part where the band is mounted may become damp due to perspiration and develop itchiness, pain, a skin rash or the like. Therefore, it is necessary to increase breathability at the band mounting site so as to restrain the site from becoming damp.

As a common method for increasing breathability at the band mounting site, multiple penetration holes may be formed in the band to expose parts of the skin to air from between band parts. With this method, ridgelines formed by the penetration holes and the back of the band come in contact with the skin. Therefore, when the band is fastened tightly, marks in the shape of the holes may be left on the skin. Also, itchiness or pain may occur along lines in the shape of the holes. Therefore, forming holes is insufficient as a measure to cope with the above problem.

Forming multiple holes may affect the appearance of the device and compromise its stylishness. Although this poses no problem for a sport watch used in exercises, appearance is important for devices such as an activity monitor used on public occasions like meetings and business negotiations, for example, or in everyday life as well.

In respect of this, JP-A-2010-110634 has no description of comfortableness in wearing wristwatch-type biological information measuring device. Meanwhile, JP-A-07-213312 discloses various forms of the band of a watch, but has no description of problems unique to a biological information measuring device, such as firmly fastening the case so as not to move, or wearing over a long period of time.

### SUMMARY

An advantage of some aspects of the invention is to provide a biological information detecting device or the like which can restrain the occurrence of itchiness, pain or a skin rash at a band mounting site.

An aspect of the invention relates to a biological information detecting device including: a biological information detector which detects biological information of a subject; and a band. The band has a first surface which is on the side of the subject when the band is worn, and has a wave-shaped outline on the side of the first surface in a cross section taken along a long-side direction of the band.

According to this aspect of the invention, the band has a wave-shaped outline on the side of the first surface in across section taken along the long-side direction of the band. Therefore, the occurrence of itchiness, pain or a skin rash at the band mounting site can be restrained.

In the aspect of the invention, the band may have the wave-shaped outline on the side of the first surface in a cross section taken along a short-side direction of the band.

With this configuration, air can enter more easily between the band mounting site and the band, thus improving breathability, or the like.

In the aspect of the invention, the band may have a first wave shape on the side of the first surface in a cross section taken along a first line in the long-side direction of the band, and a second wave shape on the side of the first surface in a cross section taken along a second line in the long-side direction of the band, and a top part of the first wave shape and a top part of the second wave shape may be shifted from each other, as viewed in a side view of the band.

With this configuration, the first surface of the band can be shaped in such a way that a plurality of wave shapes is superimposed, with their tops shifted from each other, or the like.

In the aspect of the invention, a first top part of the second wave shape may be situated between a first top part and a second top part of the first wave shape, as viewed in the side view of the band.

With this configuration, the first surface of the band can be shaped in such a way that a plurality of wave shapes is superimposed, with their tops shifted from each other, or the like.

In the aspect of the invention, a number of waves in the wave shape on the side of the first surface in the cross section taken along the short-side direction of the band may be 2 or fewer.

With this configuration, breathability can be improved further, or the like.

In the aspect of the invention, the band may include a first band having a plurality of fitting holes, and a second band having an engaging member which engages with one fitting hole of the plurality of fitting holes when the band is worn, and the first band may have the wave shape at least in a forming area for the plurality of fitting holes.

With this configuration, of the first band, the part that comes in contact with the subject's skin can be wave-shaped, or the like.

In the aspect of the invention, the biological information detecting device may include a case having the biological information detector, and the band may have a buckle or a clasp, and the band may be connected to the case and may have the wave form from the buckle or the clasp to a connecting site to the case.

With this configuration, of the second band, the part that comes in contact with the subject's skin can be wave-shaped, or the like.

### BRIEF DESCRIPTION OF THE DRAWINGS

The invention will be described with reference to the accompanying drawings, wherein like numbers reference like elements.

FIG. 1 is a perspective view showing a top side (face side) of a biological information detecting device according to an embodiment.

FIG. 2 is a perspective view showing a bottom side (back side) of the biological information detecting device according to the embodiment.

FIG. 3 is an explanatory view showing the state where the biological information detecting device is worn.

FIG. 4 is a perspective view showing the appearance of a band.

FIG. 5 is a hexahedral view of the band.

FIG. 6 is a cross-sectional view of the band.

FIG. 7 is an explanatory view of a wave shape.

FIG. 8 is an explanatory view showing a biological information detecting device having a single band.

### DESCRIPTION OF EXEMPLARY EMBODIMENTS

Hereinafter, an embodiment will be described. The embodiment described below should not unduly limit the contents of the invention described in the appended claims. Not all the configurations described in the embodiment are necessarily essential components of the invention.

### 1. Outline

In the case of a sporting device such as a running watch, a band mounting site may become damp due to perspiration and develop itchiness, pain, a skin rash or the like. Also, a wrist device with an activity measuring function or a wrist device with a pulse wave measuring function is generally worn for a long time and may be worn at the time of sleep as well. In such cases, the band is constantly in close contact with the skin and therefore the problem of itchiness and skin rash tends to occur at the site where the band is contact with the skin. Moreover, the wrist device with the pulse wave measuring function needs to be wound around the wrist more tightly than an ordinary wristwatch, in order to secure accuracy of pulse wave measurement. Therefore, the device is in closer contact with the skin and may cause pain or the like.

Thus, first of all, it is necessary to increase breathability at the band mounting site in order to prevent the site from becoming damp due to perspiration. As a common method for this, multiple penetration holes may be formed in the band to expose parts of the skin to air from between band parts. With this method, ridgelines formed by the penetration holes and the back of the band come in contact with the skin. Therefore, when the band is fastened tightly, marks in the shape of the holes may be left on the skin. Also, itchiness or pain may occur along lines in the shape of the holes. Therefore, it can be said that forming holes is insufficient as a measure to cope with the above problem of itchiness and pain when the device is worn.

Also, forming multiple holes may affect the appearance of the device and compromise its stylishness in appearance. Therefore, there is another problem that the user may feel reluctant to wear the device on public occasions like meetings and business negotiations, for example. Particularly, since the activity monitor often needs to be worn for a long period of time, it is desirable that the activity monitor has an appearance that can be suitable for any occasion.

Thus, in the embodiment, in order to solve such problems, the shape of a surface that comes in contact with the wrist, of a band of a biological information detecting device, is devised. Specifically, in the embodiment, the surface on the wrist side of the band of the biological information detecting device is formed in a continuous wave shape.

This reduces the contact area between the band and the wrist and facilitates an air flow between the band and the wrist. As a result, the dampness and wetting on the band due to perspiration can be reduced. Also, since the wave form is continuous, no angular part contacts the skin, making the occurrence of pain or itchiness less likely. In this way, according to the embodiment, the occurrence of itchiness, pain and a skin rash at the band mounting site can be restrained. Moreover, since penetration holes to secure breathability are not formed, the appearance is hardly affected and the device can be worn on any occasion.

### 2. Example of Configuration of Biological Information Detecting Device

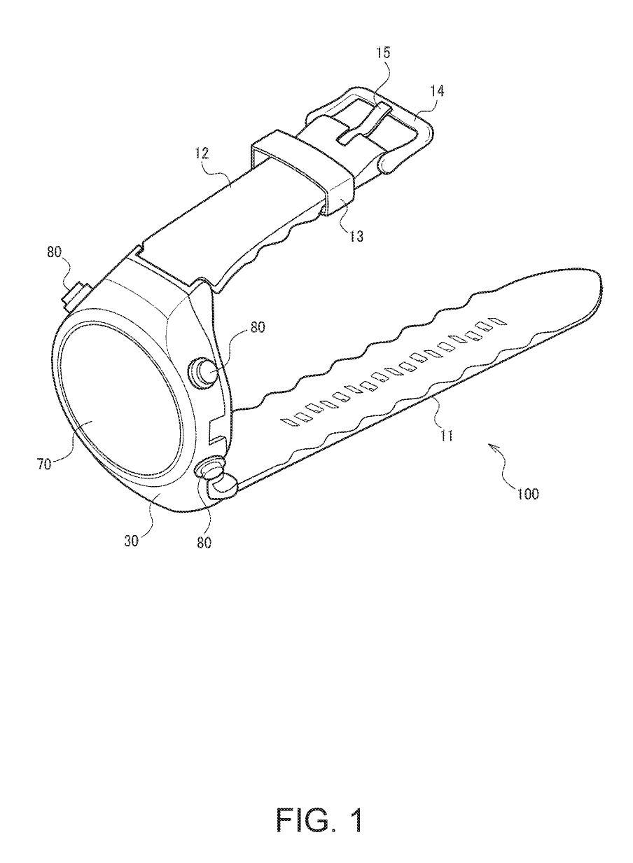

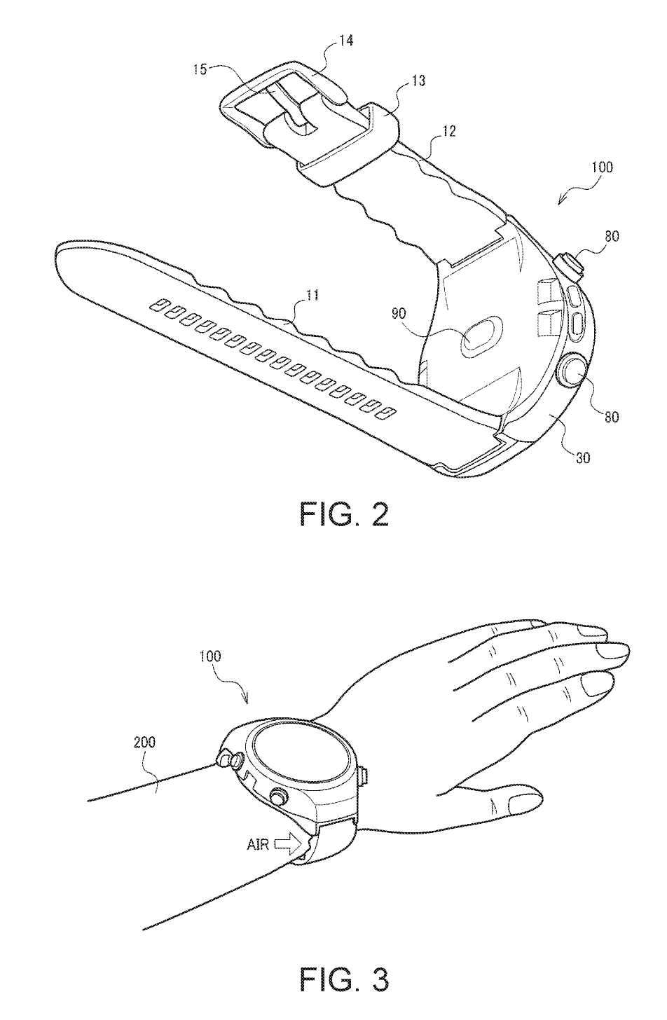

Next, an example of the configuration of a biological information detecting device 100 according to the embodiment is shown in FIGS. 1 and 2. FIG. 1 shows a perspective view of a top side (face side) of the biological information detecting device 100. FIG. 2 shows a perspective view of a bottom side (backside) of the biological information detecting device 100. The state where the biological information detecting device 100 shown in FIGS. 1 and 2 is mounted on a wrist 200 of a subject is shown in FIG. 3.

The biological information detecting device 100 in the embodiment includes a first band 11, a second band 12, a case 30, a display 70, an operator 80, and a biological information detector (sensor) 90. However, the biological information detecting device 100 is not limited to the configuration of FIGS. 1 and 2 and can be implemented with various modifications such as omitting a part of these components or adding another component.

Next, each component of the biological information detecting device 100 will be described. The first band 11 is attached to one end of the case 30 and has a plurality of fitting holes 16. The second band 12 is attached to the other end of the case 30 and has a fixing member (free ring, fixed ring) 13 and a clasp (connecting member) 14. The band 11 (12) has a first surface which is on the side of the subject when the band 11 (12) is worn. Also, in the embodiment, the band 11 (12) has a wave-shaped outline on the side of the first surface in a cross section taken along a long-side direction of the band 11 (12).

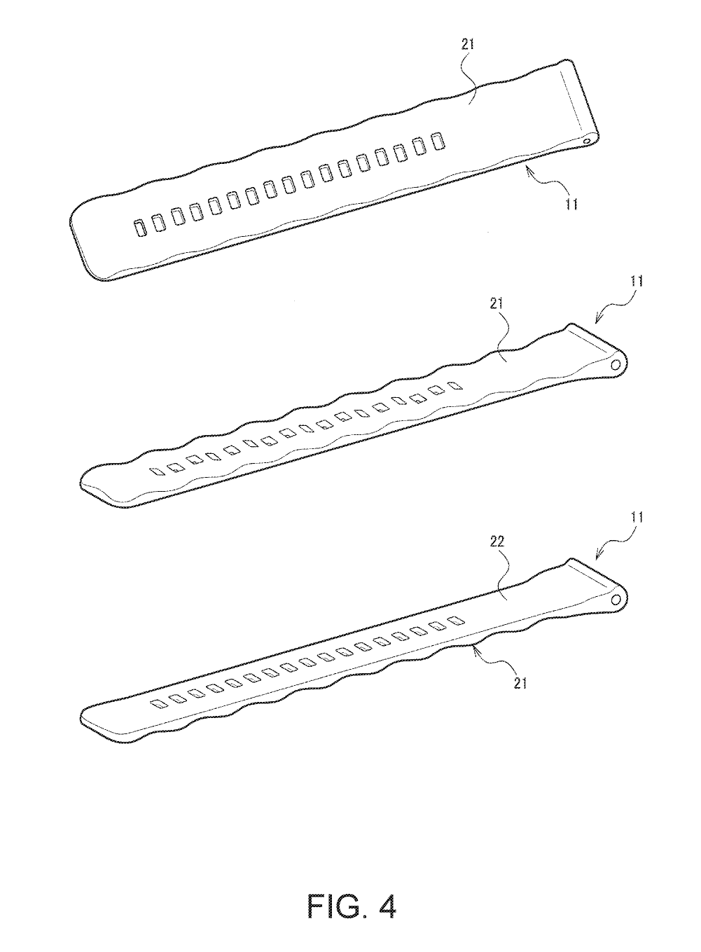

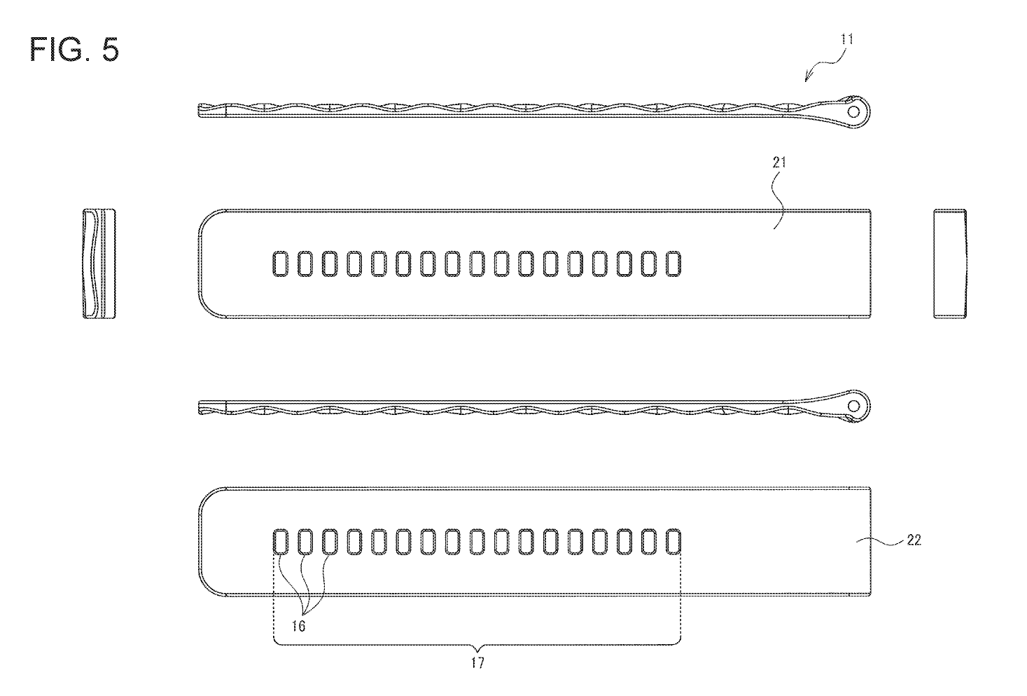

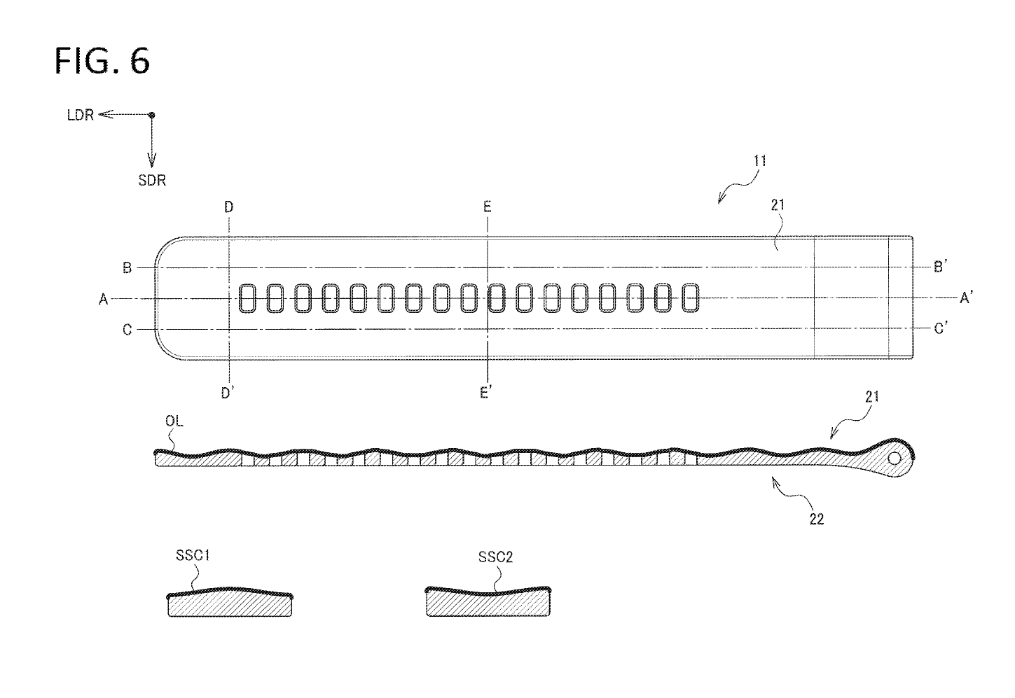

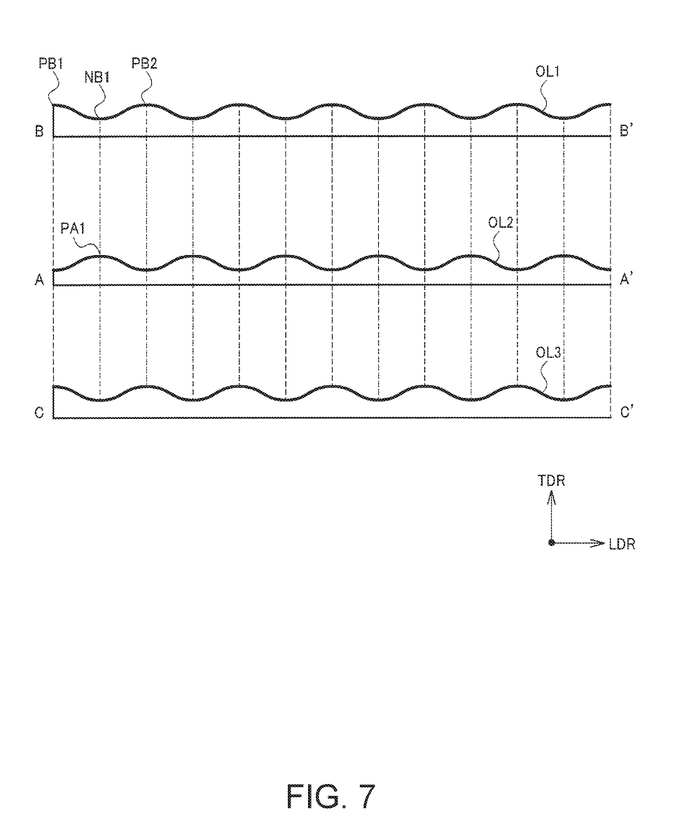

For example, FIG. 4 shows a perspective view of the appearance of the first band 11, and FIG. 5 shows a hexahedral view of the first band 11. As shown in FIGS. 4 and 5, the first band 11 has a first surface 21 which is on the side of the subject when the first band 11 is worn, and a second surface 22 which is on the outer side when the first band 11 is worn. As shown in FIG. 6, described later, the first band 11 has a wave-shaped outline on the side of the first surface 21 and a flat outline on the side of the second surface 22, in a cross section taken along the long-side direction of the first band 11. The same applies to the second band 12. Further details of the shape of the bands will be described later with reference to FIGS. 6 and 7.

If there is a step, pointed part or the like in the part of the band 11 (12) that comes in contact with the wearer's skin, it can be highly irritating to the skin and cause itchiness or a skin rash.

Meanwhile, the first surface 21 of the band 11 (12) in the embodiment is a continuous wave shape along the long-side direction (longitudinal direction) of the band 11 (12) and has no step, pointed part or the like. As described later, the wave shape also continues in a short-side direction orthogonal to the long-side direction of the band 11 (12).

Here, the wave shape is, for example, a shape formed by a plurality of single waveform shapes (single waves) laid in a line, as viewed in a cross section taken along the extending direction (long-side direction) of the band 11 (12), or as viewed in a side view orthogonal to the extending direction (long-side direction) of the band 11 (12). Each waveform shape of the plurality of waveform shapes forming the wave shape changes in a direction from the first surface 21 toward the second surface 22 over a phase range from α° to β (α<β), and changes in a direction from the second surface 22 toward the first surface 21 over a phase range from β° to γ° (β<γ). As an example, α=0°, β=180° and γ=360° may be considered. The respective waveform shapes of the plurality of waveform shapes may be different from each other. Also, a modification in which each waveform shape of the plurality of waveform shapes is a half-wave shape or the like may be employed. The wave shape may be a shape such that a contact site and a non-contact site between the band 11 (12) and the skin repeat periodically or aperiodically. Alternatively, the wave shape may be a shape such that the distance from the second surface 22 to the first surface 21 of the band 11 (12) continuously changes, periodically or aperiodically, as viewed in a cross section taken along the extending direction (long-side direction) of the band 11 (12). Moreover, the wave shape maybe a shape such that a point where the distance from the second surface 22 to the first surface 21 of the band 11 (12) is at a maximum and a point where this distance is at a minimum are alternately arranged. The wave shape is a shape that has the effect of reducing the contact area between the skin and the band 11 (12) and is less irritating to the skin. Therefore, the wave shape is less likely to cause pain or leave marks on the skin after use. People with a relatively large amount of subcutaneous fat, or people with soft skin such as women or young people, are particularly vulnerable to such marks on the skin. Therefore, the wave shape is highly effective for not leaving marks on their skin.

Also, since air AIR enters from the space formed by the wave shape, as shown in FIG. 3, breathability is improved and the accumulation of sweat between the band 11 (12) and the mounting site on the arm or the like can be prevented.

Thus, according to the embodiment, the occurrence of itchiness, pain or a skin rash at the mounting site of the band 11 (12) can be restrained.

Also, as described above, the second surface on the outer side of the band 11 (12) need not necessarily be wave-shaped and may be flatly shaped, for example. Also, there is no need to form multiple penetration holes to secure breathability. Therefore, the stylish appearance of the device is not compromised and the device can have an appearance suitable for any occasion.

Back to the description of other components, the fixing member (free ring, fixed ring) 13 fixes the first band 11 along the second band 12 in the state where the first band 11 and the second band 12 are connected together, as shown in FIGS. 1 to 3.

The clasp (clasp component, connecting member) 14 has an engaging member 15 and connects the first band 11 and the second band 12 together, as shown in FIGS. 1 and 2. The second band 12 may have a buckle instead of the clasp 14.

The engaging member 15 is fitted with one fitting hole of the plurality of fitting holes 16 when the device is worn. The engaging member 15 is formed by a metal rod or a plastic rod, as shown in FIG. 1, for example.

Each fitting hole of the plurality of fitting holes 16 is a hole penetrating the first band 11 in the direction of its thickness and provided at a predetermined interval in the first band 11, as shown in FIG. 1. When the biological information detecting device 100 is mounted on the subject, the engaging member 15 is inserted in one fitting hole of the plurality of fitting holes 16, thus enabling the second band 12 engaged with the first band 11. Moreover, by selecting a fitting hole in which the engaging member 15 is to be inserted, of the plurality of fitting holes 16, it is possible to adjust the length of the band tightened at the mounting site on the subject. However, the engaging member 15 is not limited to a single rod as shown in FIG. 1. For example, the engaging member 15 may have a plurality of rods so that, when the biological information detecting device 100 is mounted on the subject, the individual rods may be inserted in different fitting holes of the plurality of fitting holes, thus engaging the second band 12 with the first band 11. The engaging member 15 may also be a forceps member which pinches (or nips) the first band 11.

The case 30 is provided with the biological information detector (sensor) 90 for detecting biological information of the subject.

The biological information detector 90 has a light emitter, not shown, a light receiver, not shown, and a detection window, and detects biological information of the subject in the state of being pressed against the subject. Specifically, light emitted from the light emitter is cast on the subject via the detection window, and the light reflected from the subject becomes incident on the light receiver via the detection window again. The biological information detector 90 detects biological information of the subject on the basis of the reflected light received by the light receiver. Here, the detection window is formed of a transparent member or a semitransparent member which transmits the light emitted from the light emitter and the reflected light reflected from the subject. More specifically, the detection window is formed of a transparent plastic or the like, for example. When the device is mounted on the subject, the detection window plays the role of physically separating the light emitter and the light receiver from the subject. The light receiver can be implemented by a light receiving element such as a photodiode. The light emitter can be implemented by a light emitting element such as an LED.

The display 70 displays various kinds of information. The functions of the display 70 can be implemented by a liquid crystal display, organic EL display, electronic paper or the like.

The operator 80 accepts an operation by the user. The operator 80 can be implemented by a button, switch, touch panel or the like.

### 3. Detailed Description of Shape of Band

Next, the shape of the band will be described in detail with reference to FIGS. 6 and 7. FIG. 6 shows a cross-sectional view in each direction of the first band 11. While the shape of the first band 11 will be described below, a similar shape can be applied to the second band 12 as well.

As shown in FIG. 6, the band 11 has a wave-shaped outline on the side of the first surface 21 in a cross section taken along a long-side direction LDR of the band 11 (for example, A-A′ cross section).

Here, the long-side direction refers to a direction from one end of the band 11 connected to the case 30 toward the other end, or its opposite direction. In the example of FIG. 6, the direction LDR is the long-side direction.

The outline (ridgeline) in a cross section refers to a line tracing the outline (outer frame or outer circumference) of a cross section when the cross section is viewed in a side view. The outline of the shape situated behind the cross section is not the outline of the cross section. As an example, FIG. 6 shows an A-A′ cross section taken along the long-side direction LDR of the band 11. The outline on the side of the first surface 21 in the cross section of the band 11 is a line indicated by a bold line OL. This outline OL is wave-shaped.

As shown in FIG. 6, the band 11 has a wave-shaped outline on the side of the first surface 21 in a cross section taken along a short-side direction SDR of the band 11 (for example, D-D′ cross section and E-E′ cross section).

Here, the short-side direction is a direction orthogonal to (intersecting with) the long-side direction, or its opposite direction. In the example of FIG. 6, the direction SDR is the short-side direction.

For example, FIG. 6 shows a D-D′ cross section taken along the short-side direction SDR and an E-E′ cross section taken along the short-side direction SDR. The outline on the side of the first surface 21 in the D-D′ cross section is SSC1 indicated by a bold line. This outline SSC1 is also wave-shaped. Similarly, the outline on the side of the first surface 21 in the E-E′ cross section is SSC2 indicated by a bold line. This outline SSC2 is also wave-shaped.

In this way, the first surface 21 of the band 11 is wave-shaped not only in the long-side direction LDR of the band 11 but also in the short-side direction SDR of the band 11. Therefore, the first surface 21 of the band 11 can be shaped in such a way that a plurality of wave shapes is superimposed, with their tops shifted from each other, as described later. As a result, air can enter more easily between the mounting site of the band 11 and the band 11, thus enabling improvement in breathability or the like.

In the embodiment, the fitting holes can be formed, including the tops of the outline. As an example, the fitting holes can be provided at the tops on the side of the second surface 22 of the band 11, that is, at the tops farther from the skin (extremum points). This configuration enables further improvement in breathability.

Next, the shape in which a plurality of wave shapes is superimposed, with their tops shifted from each other, will be described specifically with reference to FIG. 7. FIG. 7 schematically shows the A-A′ cross section, B-B′ cross section and C-C′ cross section of the band 11 shown in FIG. 6.

The band 11 in the embodiment has a first wave shape on the side of the first surface 21 in a cross section taken along a first line in the long-side direction of the band 11, and a second wave shape on the side of the first surface 21 in a cross section taken along a second line in the long-side direction of the band 11. Top parts of the first wave shape and top parts of the second wave shape are shifted from each other, as viewed in a side view of the band 11.

That is, specifically, if the first line in the long-side direction LDR is a line B-B′ and the second line in the long-side direction LDR is a line A-A′ in the example of FIG. 7, an outline OL1 on the side of the first surface 21 in a cross section taken along the first line B-B′ has the first wave shape. Similarly, an outline OL2 on the side of the first surface 21 in a cross section taken along the second line A-A′ has the second wave shape. In this case, in the band 11, top arts (PB1 and PB2) of the first wave shape and a top part PA1 of the second wave shape are shifted from each other, as viewed in the side view of the band 11. Similarly, top parts in the B-B′ cross section and top parts in the cross section C-C′ are shifted from each other.

It can also be said that the first top part PA1 of the second wave shape is situated between the first top part PB1 and the second top part PB2 of the first wave shape, as viewed in the side view of the band 11.

The top part in this example refers to a predetermined range including a top where a switching from ascent to descent takes place in the wave shape.

Thus, the first surface 21 of the band 11 can be shaped in such a way that a plurality of wave shapes is superimposed, with their tops shifted from each other, or the like.

By optimizing the number of waves in the wave shape, its wavelength, amplitude and the like, it is possible to further improve breathability.

For example, it is desirable that the number of waves in the wave shape on the side of the first surface in the cross section taken along the short-side direction of the band 11 is 2 or fewer.

The wave shape is formed, for example, by having an aperiodic waveform W repeated periodically, as described above. In this case, the number of waves in the wave shape is the number of waveforms W included in the wave shape. Specifically, it is preferable that the number of waves in the wave shape on the side of the first surface 21 in the cross section taken along the short-side direction of the band 11 is 1. For example, in FIG. 6 described above, the number of waves in the wave shape on the side of the first surface 21 in the D-D′ cross section or the E-E′ cross section is 1. In this case, it can be said that the wave shape itself is an aperiodic waveform.

The wavelength of the wave shape refers to the distance between top parts in the wave shape, in the long-side direction or the short-side direction. For example, in the example of the B-B′ cross section in FIG. 7, the wavelength of the wave shape is the distance from the top part PB1 to the top part PB2 in the long-side direction LDR.

The amplitude of the wave shape refers to the distance between a top part and a bottom part in the wave shape, in the direction of the thickness of the band 11. For example, in the example of the B-B′ cross section in FIG. 7, the amplitude of the wave shape is the distance between the top part PB1 and a bottom part NB1, in a thickness direction TDR.

This enables further improvement in breathability, or the like.

As shown in FIG. 5 described above, the first band 11 may have a wave shape at least in a forming area 17 for the plurality of fitting holes 16.

Here, the forming area 17 for the plurality of fitting holes 16 refers to an area from the position of a fitting hole that is the nearest to the one end of the first band 11, of the plurality of fitting holes 16, to the position of a fitting hole that is the nearest to the other end of the first band 11, of the plurality of fitting holes 16. In the first band 11, the forming area 17 for the plurality of fitting holes 16 is most likely to be in contact with the subject's skin.

Thus, the part that comes in contact with subject's skin, of the first band 11, can be wave-shaped or the like.

As shown in FIGS. 1 and 2 described above, in the second band 12, a wave shape may be formed from the clasp (or buckle) 14 to the connecting site to the case 30.

Thus, the part that comes in contact with the subject's skin, of the second band 12, can be wave-shaped or the like.

As described above, according to the embodiment, simply changing the shape of the band without adding a special material or component can achieve the effect of restraining itchiness, pain, a skin rash or the like when the device is worn.

In the embodiment, it is also possible to enhance various effects by carrying out the following processing on the band.

For example, if a material with a high heat radiation ability (for example, silicone or the like) is used as the material of the band, the effect of reducing dampness or wetting due to perspiration can be increased. Also, if a low skin-irritating material (for example, silicone or the like) is used as the material of the band, the itchiness and skin rash can be reduced.

Also, if the surface of the band is coated with a material which lowers a frictional resistance value (for example, a fluorine material or the like), stickiness on the surface of the band can be reduced, thus enabling a reduction itchiness and skin rash.

Fine irregularities may be formed on the surface of the band by surface texturing on the surface of the wave shape of the band. Here, surface texturing refers to a kind of surface treatment in which crease patterns (fine irregularities) are physically formed. This enables a further increase in the contact with air and a reduction in dampness and wetting, or the like.

Alternatively, fine waves may be formed on the surface of the wave shape of the band. For example, a fractal shape may be formed. This enables a further reduction in dampness and wetting.

Also, if a wave shape is formed on the back side (wrist side) of the case as well as on the band, an increase in the effect can be expected.

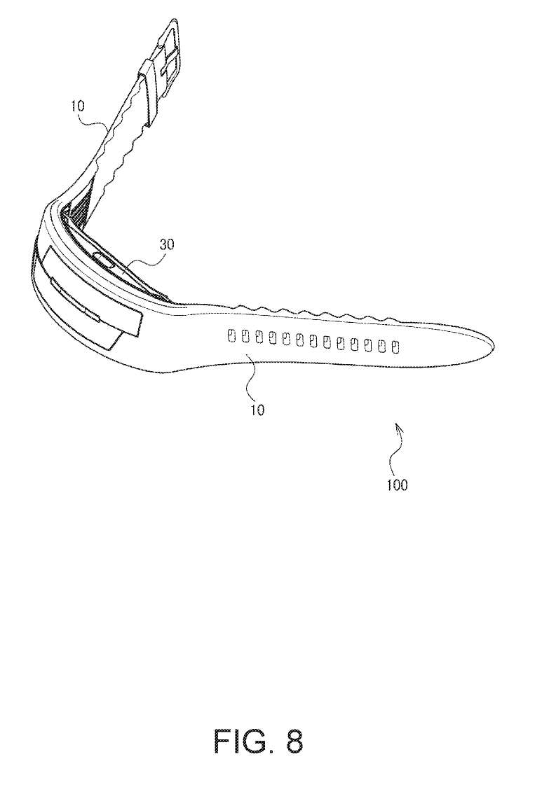

The embodiment described up to this point can be applied not only to the biological information detecting device 100 having the first band 11 and the second band 12 as shown in FIG. 1, but also to a biological information detecting device 100 having only a single band 10 as shown in FIG. 8. The biological information detecting device 100 shown in FIG. 8 has a case 30 provided on the back side of the band 10.

While the embodiment has been described in detail above, a person skilled in the art can readily understand that various modifications can be made without substantially departing from the new matters and advantageous effects of the invention. Therefore, all such modifications are included in the scope of the invention. For example, a term described along with a different term with a broader meaning or the same meaning at least once in the specification or drawings can be replaced with the different term at any point in the specification or drawings. Also, the configurations and operations of the biological information detecting device are not limited to those described in the embodiment and can be implemented with various modifications.

What is claimed is:

1. A biological information detecting device comprising:
a biological information detector which detects biological information of a subject; and
a band;
wherein the band has a first surface which is on the side of the subject when the band is worn, and has a wave-shaped outline on the side of the first surface in a cross section taken along a long-side direction of the band.

2. The biological information detecting device according to claim 1, wherein
the band has the wave-shaped outline on the side of the first surface in a cross section taken along a short-side direction of the band.

3. The biological information detecting device according to claim 2, wherein
the band has
a first wave shape on the side of the first surface in a cross section taken along a first line in the long-side direction of the band, and
a second wave shape on the side of the first surface in a cross section taken along a second line in the long-side direction of the band, and
a top part of the first wave shape and a top part of the second wave shape are shifted from each other, as viewed in a side view of the band.

4. The biological information detecting device according to claim 3, wherein
a first top part of the second wave shape is situated between a first top part and a second top part of the first wave shape, as viewed in the side view of the band.

5. The biological information detecting device according to claim 2, wherein
a number of waves in the wave shape on the side of the first surface in the cross section taken along the short-side direction of the band is 2 or fewer.

6. The biological information detecting device according to claim 1, wherein
the band includes
a first band having a plurality of fitting holes, and
a second band having an engaging member which engages with one fitting hole of the plurality of fitting holes when the band is worn, and
the first band has the wave shape at least in a forming area for the plurality of fitting holes.

7. The biological information detecting device according to claim 1, comprising
a case having the biological information detector,
wherein the band has a buckle or a clasp, and
the band is connected to the case and has the wave form from the buckle or the clasp to a connecting site to the case.
